(19) United States
(12) Patent Application Publication
(10) Pub. No.: US 2025/0042633 A1
(43) Pub. Date: Feb. 6, 2025

(54) SINGLE-USE PACKAGING

(71) Applicant: NOTPLA LIMITED, London (GB)

(72) Inventors: Pierre-Yves PASLIER, London (GB); Rodrigo Garcia GONZALEZ, London (GB)

(21) Appl. No.: 18/709,176

(22) PCT Filed: Nov. 11, 2022
(86) PCT No.: PCT/GB2022/052864

§ 371 Date: May 10, 2024

(30) Foreign Application Priority Data
Nov. 12, 2021 (GB) 2116334.0

(51) Int. Cl. B65D 65/46; C08L 5/04

(52) U.S. Cl. CPC B65D 65/466; C08L 5/04

(57) ABSTRACT

The invention relates to a water-based viscous liquid encapsulated within a membrane, wherein the viscous liquid has a water content from 2 wt % to 55 wt %, and wherein the membrane comprises alginate and calcium ions. The Invention also relates to methods of preparing the same and uses thereof.

### FIELD OF INVENTION

The present invention relates to water-based viscous liquids encapsulated in an alginate membrane, and methods of producing the same, for packaging consumer products, such as edible and cosmetic goods.

### BACKGROUND

Plastic packaging is used in abundance globally and large quantities of plastic end up being disposed of in landfill or incinerated, thus contributing to environmental damage such as marine pollution. The impact of plastic packaging on the environment is huge. It has been estimated that the societal cost of plastic produced just in 2019 to be $3.7 trillion (WWF, 2021) and that €13 billion a year of damage to global marine ecosystems is caused by plastic. Further, 80-85% of marine litter found on Europe's beaches is plastic (EU Single Use Plastics Directive, ISBN 978-92-76-12903-5).

Currently, a major cause of plastic not being recycled is the difficulty in recycling particular types of plastic, such as the thin plastic films used in packaging food products or other consumer products. There is therefore a need to develop environmentally friendly alternatives to plastic packaging.

Alginate is a water-soluble biopolymer that is extracted from brown seaweed. It is a well-known and valuable natural material which has a wide variety of practical applications. Alginate is edible and was first utilised for this property by Unilever in the 1950s and brought to modern cuisine via a process called spherification. Spherification is a culinary process that uses sodium alginate and either calcium chloride or calcium gluconate lactate to form a spherical membrane around a liquid to form squishy spheres, which visually and texturally resemble roe. It is a technique that is now commonly used in the food industry to add texture and an exciting “popping” sensation in a food/drink consumer's mouth. However, as these uses are designed to be eaten soon after formation they are not stable for long periods of time and are susceptible to liquid leakage.

Additionally whilst there has been speculation about alginate's utility for encapsulating cosmetic products, to date it has not been successfully done.

The enhancement and development of alginate properties, specifically gelling, film-forming and stabilising characteristics alongside mechanical properties for the purpose of long-term packaging and storage of consumer products are of great interest to reduce the environmental damage that single-use plastics are causing. Alginate membranes biodegrade very quickly in the natural environment without the need for industrial composting conditions or niche microorganisms so are an excellent alternative material to single-use and non-biodegradable or non-recyclable plastics.

### SUMMARY OF THE INVENTION

The present invention seeks to address the problem outlined above by developing alginate membranes to encapsulate water-based viscous liquids. This results in a cost effective, environmentally sustainable solution for packaging and storing viscous liquid consumer products, in particular as an alternative to plastic packaging such as sachets or tubes. The inventors have surprisingly found that a viscous liquid with particular properties (particularly those that have a water content from 2 wt % to 55 wt %) can be encapsulated in an alginate/calcium membrane and remain stably stored for significant periods of time without the liquid permeating out of the membrane. This allows the biodegradable alginate membrane to replace plastic packaging of such viscous liquid consumer products.

Accordingly, a first aspect of the invention is a water-based viscous liquid encapsulated within a membrane, wherein the viscous liquid has a water content from 2 wt % to 55 wt %, and wherein the membrane comprises alginate and calcium ions.

A second aspect of the invention is a method of preparing a water-based viscous liquid encapsulated within a membrane, said method comprising the steps of:
(i). providing an aqueous alginate solution free from calcium ions;
(ii). providing a viscous liquid having a water content from 2 wt % to 55 wt % and calcium ions;
(iii). contacting the viscous liquid of step (ii) with the alginate solution of step (i) thereby forming the water-based viscous liquid encapsulated within a membrane.

A third aspect of the invention is the use of the water-based viscous liquid encapsulated within a membrane as provided in the first aspect or the container of the fourth aspect to:
(i). package and/or store the viscous liquid; and/or
(ii). transport the viscous liquid; and/or
(iii). provide a single dose of the viscous liquid to a user.

A fourth aspect of the invention is a container comprising at least one of the water-based viscous liquid encapsulated within a membrane according to the first aspect of the invention.

### DESCRIPTION OF THE FIGURES

FIG. 1 illustrates a Spherical shape of a water-based viscous liquid (1) encapsulated within a membrane (2).

FIG. 2 illustrates a Ellipsoid shape of a water-based viscous liquid (1) encapsulated within a membrane (2).

FIG. 3 illustrates a Ovoid shape of a water-based viscous liquid (1) encapsulated within a membrane (2).

FIG. 4 illustrates an Oblong shape of a water-based viscous liquid (1) encapsulated within a membrane (2).

FIG. 5 illustrates a Drop shape of a water-based viscous liquid (1) encapsulated within a membrane (2).

FIG. 6 illustrates a Long tall drop shape of a water-based viscous liquid (1) encapsulated within a membrane (2).

FIG. 7A-F illustrates a group of units of water-based viscous liquids encapsulated within a membrane (1) conjoined in a plane with or without an external membrane (2).

FIG. 8A-F illustrates a group of units of water-based viscous liquids encapsulated within a membrane (1) conjoined in a three dimensional configuration with or without an external membrane (2).

FIG. 9A-G illustrates different units of water-based viscous liquids encapsulated within a membrane (1) with a stalk (2) with different shapes, individually or as a grouping.

FIG. 10A-F illustrates a group of units of water-based viscous liquids encapsulated within a membrane (1) conjoined with other units of different sizes and or contents (2).

FIG. 11A-C illustrates different groups of units of water-based viscous liquids encapsulated within a membrane conjoined with others in a radial configuration.

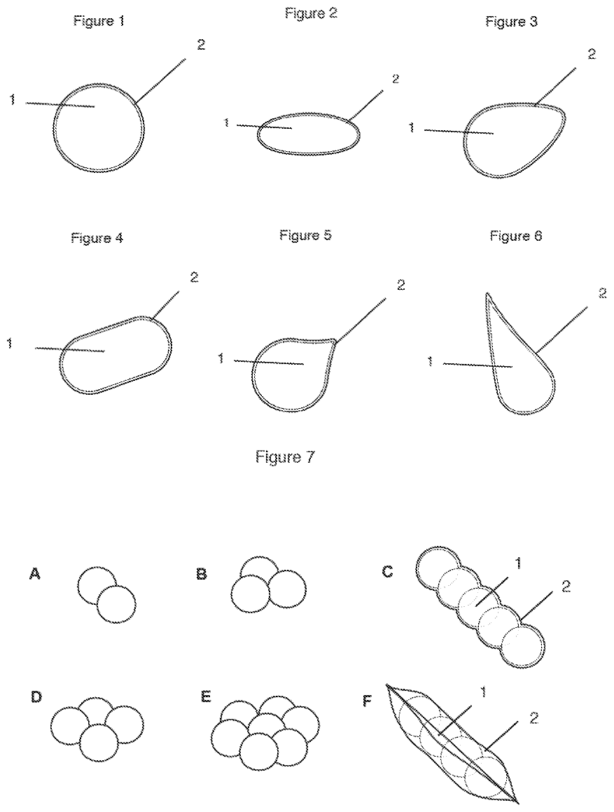

### DETAILED DESCRIPTION OF THE INVENTION

Unless indicated otherwise, all technical and scientific terms used herein will have their common meaning as understood by one of ordinary skills in the art to which this invention pertains.

The term “comprising” or variants thereof will be understood to imply the inclusion of a stated element, integer or step, or group of elements, integers or steps, but not the exclusion of any other element, integer or step, or group of elements, integers or steps.

The term “consisting” or variants thereof is to be understood to imply the inclusion of a stated element, integer or step, or group of elements, integers or steps, and the exclusion of any other element, integer or step or group of elements, integers or steps.

As used herein, the term “viscous liquid” refers to a flowable product that may have a viscosity of up to 250,000 cps. As such, the term “viscous liquid” is intended to encompass both low viscosity products such as liquid coffee and higher viscosity products such as pastes, slurries, suspensions, creams and sauces.

Viscosity describes a liquid's thickness, more specifically its resistance to flow and is reported in centipoise (cP). Viscosity ranges for liquid categories are: thin (1-50 cP), nectar-like (51-350 cP), honey-like (351-1,750 cP), and spoon-thick (greater than 1,750 cP). Because the thickness of most consumable food based liquids changes as a function of its flow rate, viscosity measures are typically reported at a specific shear rate. These liquids are described as “shear-thinning” in that they decrease in viscosity as their flow rate increases.

The term “water-based viscous liquid” refers to a viscous liquid that comprises water. The inventors have found that the viscous liquid requires at least 2 wt % water content in order to form the alginate membrane. Viscous liquids with no water content do not dissolve the calcium salts and thus do not react with the alginate to form the membrane.

The inventors have additionally found that the viscous liquid requires at most 55 wt % water content in order for the viscous liquid to not permeate through the membrane, and thus remain stable over time.

“Viscosity” is typically measured at room temperature (e.g. 20° C.). The viscosity of certain liquids may be dependent on shear rate (e.g. shear thinning fluids). The term “viscous liquid” as used herein is intended to cover shear rate-independent fluids which have a viscosity of up to 100,000 cps as well as shear thinning fluids which have a viscosity of up to 100,000 cps at a high shear rate (e.g. a shear rate of 10 s⁻¹ or 100 s⁻¹). Where viscosities of liquid products are discussed herein, the values mentioned are to be interpreted as viscosity at a high shear rate (e.g. a shear rate of 10 s⁻¹ or 100 s⁻¹) when applied to shear thinning fluids. The values herein are measured by dynamic (absolute) viscosity measurements. Dynamic viscosity is also known as absolute viscosity and most often relates to non-Newtonian fluids. It refers to the fluid's internal resistance to flow when force is applied. The dynamic viscosity can be measured using apparatus such as the following:
Falling piston viscometers: Falling piston viscometers use the force created by a falling piston to measure viscosity. They measure dynamic viscosity, because stress is applied to the fluid.
Rotational viscometers: A rotational viscometer measures how much torque is required to turn a spindle immersed in a fluid. The spindle applies stress to the fluid, resulting in a measurement of dynamic viscosity.
Falling ball viscometers: A falling ball viscometer measures the force required for a ball to fall through a fluid. The ball applies stress to the fluid, giving a measurement of dynamic viscosity. Patented in 1932 by Fritz Höppler, the falling ball viscometer was actually the first type of viscometer to measure dynamic viscosity.
Vibrational viscometers: Vibrational viscometers measure the resistance of a fluid to vibration. Since the vibration constitutes a force being applied to the fluid, these viscometers measure dynamic viscosity (Johnson, J. F. et al, 1975, Determination of Viscosity of Food Systems. Theory, Determination and Control of Physical Properties of Food Materials, 25-38).

The term “encapsulated” means being enclosed or surrounded by (e.g. In a membrane).

The term “membrane” means a thin layer, film or skin forming a barrier or lining. The membranes of the present invention are continuous (due to the spherification technique used to make them) and thus do not comprise a mechanically or otherwise sealed edge (i.e. a ‘seal line’) which would form with other methods of encapsulation (such as with filling a pre-made membrane with the desired liquid, then sealing the membrane). An advantage of not having such a seal line is that leakage through imperfectly sealed points is avoided. Additionally, by not having a seal line, the manufacturing process is seamless and uniform, as the encapsulation occurs in one step that is easily repeatable and tuneable. The encapsulation process used herein is faster, uses less complex machinery (as no joining of the membrane is required), and there is overall less wastage compared to other methods of encapsulation.

The encapsulated water-based viscous liquids according to the invention are also referred to as “pearls” herein.

“wt %” is a common abbreviation in the art to mean the “weight %” with respect to the total weight of the article/material referred to.

The present invention provides a water-based viscous liquid encapsulated within a membrane, wherein the viscous liquid has a water content from 2 wt % to 55 wt %, and wherein the membrane comprises alginate and calcium ions.

In some embodiments, the viscous liquid has a water content from 5 wt % to 55 wt %, preferably 5 wt % to 50 wt %, more preferably from 5 wt % to 45 wt %, more preferably from 5 wt % to 40 wt %, yet more preferably from 5 wt % to 35 wt %, even more preferably from 5 wt % to 30 wt %, still more preferably from 5 wt % to 25 wt %, such as from 5 wt % to 20 wt %, for example from 5 wt % to 15 wt %. Suitably the viscous liquid has a water content of greater than 10 wt % up to any of the aforementioned upper limits.

Suitably, the viscous liquid has a water content of greater than 2 wt %, such as greater than 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, or 20 wt %. Suitably, the viscous liquid has a water content of less than 55%, such as less than 50 wt %, 49 wt %, 48 wt %, 47 wt %, 46 wt %, 45 wt %, 44 wt %, 43 wt %, 42 wt %, 41 wt %, 40 wt %, 39 wt %, 38 wt %, 37 wt %, 36 wt %, 35 wt %, 34 wt %, 33 wt %, 32 wt %, 31 wt %, 30 wt %, 29 wt %, 28 wt %, 27 wt %, 26 wt %, or 25 wt %. Any of the aforementioned lower or upper limits of water content may be combined with each other, and are herein disclosed.

In some embodiments, the water-based viscous liquid comprises calcium salts in an amount from 1 wt % to 15 wt %, preferably 1 wt % to 10 wt %, preferably 2 wt % to 10 wt %, more preferably 2 wt % to 8 wt %, more preferably 2 wt % to 5 wt % with respect to the total liquid weight. In alternative preferred embodiments, the water-based viscous liquid comprises calcium salts in an amount from 1 wt % to 5 wt %, such as 1 wt % to 3 wt %.

Suitably, the viscous liquid has a viscosity from 20,000 to 250,000 cps, preferably 20,000 to 100,000. Alternatively, the viscous liquid has a viscosity of 1 to 100,000 cps, more preferably 100 to 4,000 cps. Alternatively the viscous liquid has a viscosity from 1 to 20,000 cps.

Suitably, the viscous liquid has a viscosity of greater than 5 cps, such as greater than cps, 50 cps, 100 cps, 200 cps, 500 cps, 1,000 cps, 1,500 cps, 2,000 cps, 2,500 cps, 3,000 cps, 5,000 cps, 10,000 cps, 15,000 cps, or 20,000 cps. Suitably, the viscous liquid has a viscosity of less than 100,000 cps, such as less than 90,000 cps, 80,000 cps, 70,000 cps, 60,000 cps, 50,000 cps, 40,000 cps, 30,000 cps, 20,000 cps, 15,000 cps, 10,000 cps or 5,000 cps. Any of the aforementioned lower or upper limits of viscosity may be combined with each other, and are herein disclosed.

The pearls are made by the well-known spherification process which involves injecting the viscous liquid into a bath of alginate solution. In order for this process to work and result in the alginate membrane forming around the viscous liquid, the viscosity of the viscous liquid must be greater than that of the alginate bath. Therefore, in some embodiments, the viscous liquid comprises a thickening agent (also referred to as a “thickener”), which increases the viscosity of the viscous liquid. This is useful if the viscous liquid (i.e. the consumer product) has a viscosity lower than or similar to the alginate bath's viscosity.

In some embodiments, the alginate bath has a viscosity of 50 to 5000 cps, preferably 50 to 1000 cps, such as 50 to 500 cps, more preferably 100 to 400 cps.

Suitably, the thickener comprises a natural or vegetable gum, such as a natural polysaccharide from the glucomannan group or a thickener derived from seaweed.

In some embodiments, the thickener comprises one of maltodextrin, starch, modified starch, cellulose gel, guar gum, tara gum, carrageenan, gum tragacanth, locust bean gum, microcrystalline cellulose, pectin, gellan gum, glucumannans, succinoglucan, konjac glucomannan, agar, pectin, xanthan gum, mesquite gum, gum arabica, glycerol, or mixtures of two or more thereof.

In preferred embodiments, the thickener comprises maltodextrin, glycerol, guar gum, pectin, agar, xanthan gum, starch (such as modified food starch, particularly cornstarch) or a mixture of two or more thereof. Most preferably, the thickening agent comprises starch, and/or glycerol and/or maltodextrin.

Suitably, the plasticiser comprises one of dihydric acids (such as ethylene glycol, propylene glycol), trihydric alcohols (such as glycerol, polyglycerol), sugar alcohols (such as sorbitol, mannitol, maltitol, xylitol), sacchariflcation products of reduced starch (such as glucose, fructose, galactose and xylose), disaccharides (such as saccharose, maltose and lactose), amino acids (such as proline, glycine, alanine), amino acids in combination with polyols, short chain fatty acids (such as capric acid, caproic acid), or a mixture of two or more thereof.

Suitably the viscous liquid comprises greater than 1 wt % of glycerol, such as greater than 5 wt %, 10 wt %, 20 wt %, 30 wt %, 40 wt %, 50 wt %, 60 wt %, 70 wt % and/or the viscous liquid comprises less than 90 wt % of glycerol, such as less than 80 wt %, 70 wt %, 60 wt %, 50 wt %, 40 wt %, 30 wt %.

In some embodiments, the calcium ions are sourced from one of calcium gluconolactate, calcium chloride, calcium iodide, calcium bromide, calcium fluoride, calcium hydroxide, calcium citrate, calcium acetate, calcium gluconate, or calcium carbonate, or a combination of two or more thereof, preferably calcium gluconolactate and/or calcium chloride.

In some embodiments, the viscous liquid comprises a non-ionic surfactant, preferably a non-ionic surfactant with a Hydrophile-Lipophile Balance (HLB) value of 0 to 20. Suitably, the non-ionic surfactant comprises, such as consists of, one of a bioderived surfactant (such as sugar surfactants such as sucrose esters, methyl glycoside esters, ethyl glycoside esters, N-methyl-glucamides or sorbitan esters, or alkyl polyglucosides (APGs)) or a mixture of two or more thereof.

Suitably, the non-ionic surfactant comprises, such as consists of, one of lauryl glucoside, decyl glucoside, glycol distearate, glycerol monostearate, propylene glycol isostearate, a Tween® (such as Tween® 20, 40, 60, 80), a Span™ (such as Span™ 20, 40, 60, 80, 85) or a Polysorb® (such as Polysorb®85/70/00), polysorbate (such as polysorbate 20, 40, 60, 80) or lecithin, or a mixture of two of more thereof.

Suitably, the non-ionic surfactant comprises, such as consists of, APGs, preferably lauryl glucoside and/or decyl glucoside.

In some embodiments, the non-ionic surfactant comprises a bioderived surfactant, preferably non petrochemically derived.

In some embodiments, the viscous liquid comprises an amphoteric surfactant. Suitably, the amphoteric surfactant comprises one or more of cocoamidopropyl betaine, lauryl betaine, betaine citrate, sodium lauroamphoacetate, sodium hydroxymethylglycinate, (carboxymethyl)dimethyloleylammonium hydroxide, (carboxylatomethyl)dimethyl(octadecyl)ammonium or a mixture of two or more thereof, preferably cocoamidopropyl betaine.

Suitably, the amphoteric surfactant may comprise a blo-derived, biodegradable, non-petrochemical derived surfactant, such as an algal betalne surfactant made from renewable microalgae oil. In particular embodiments, the algal betaine surfactant is Dehyton® AO 45 (BASF). Such a betaine surfactant may be used as an alternative to cocoamidopropyl.

In some embodiments, the viscous liquid and/or the membrane comprise(s) one or more of an additive, diluent, food additive, food flavouring, fragrance or preservative.

In preferred embodiments, the membrane comprise(s) an additive. The additives provide additional functional/performance effects e.g. Increased plasticity, increased mechanical strength or improved permeability to water. This results in increased stability of the encapsulated viscous liquid and even longer storage times without significant permeation and loss of content. Suitably, the additive is one of or a mixture of two or more of silicone dioxide, kaoline, annatto, bamboo fibre, silk amino acid, chitosan, 1,3-butylene glycol, acacia, acetic and fatty acid esters of glycerol, acetone, acetylated distarch adipate, acetylated monoglycerides, acid-treated starch, agar, alginic acid, alkaline-treated starch, anoxomer, ascorbic acid, ascorbyl palmitate, ascorbyl stearate, azodicarbonamide, beeswax, bleached starch, bone phosphate, brominated vegetable oil, calcium acetate, calcium alginate, calcium aluminum silicate, calcium ascorbate, calcium benzoate, calcium bromate, calcium carbonates, calcium chloride, calcium citrate, calcium dihydrogen phosphate, calcium disodium ethylenediamine-tetraacetate, calcium DL-malate, calcium ferrocyanide, calcium gluconate, calcium hydrogen sulfite, calcium hydroxide, calcium iodate, calcium lactate, calcium lactate gluconate, calcium lactobionate, calcium peroxide, calcium phosphate, calcium polyphosphates, calcium propionate, calcium pyrophosphatecalcium salts of fatty acids, calcium silicate, calcium sorbate, calcium stearate, calcium stearoyl lactylate, calcium sulfate, calcium tartrate, calciumiodiate, candelilla wax, carbamide, carbon dioxide, carnauba wax, carob bean gum, carrageenan, castor oil, cellulose gum, celluloses, choline salts and esters, citric acid, citric and fatty acid esters of glycerol, crosslinked sodium carboxymethylcellulose, cupric sulfate, D-alpha-tocopherol, dammar gum, decanoic acid, dedesoxycholic acid, dedextrins, dextrin ethyl cellulose, dextrose, diacetyltartaric acid esters of mono- and diglycerides of fatty acids, diammonium hydrogen phosphate, dicalcium pyrophosphate, diethyl pyrocarbonate, ethyl alcohol, ethyl cellulose, ethyl hydroxyethyl cellulose, ethyl p-hydroxybenzoate, ethyl protocatechuate, ethylene dichloride, esters of glycerol and thermally oxidized soy bean fatty acids, ethoxylated mono- and diglycerides, ethyl hydroxyethyl cellulose, formic acid, gellan gum, gelatin, genipin, gibberellic acid, glucono delta-lactone, glycerin, glycerol, glycerol ester of wood rosin, guaiac resin, guar gum, gum acacia, gum arabic, gum ghatti, gum guaiac, heptylparaben, peroxide derivatives, hydrogen peroxide, hydroxylated lecithin, hydroxypropyl cellulose, hydroxypropyl distarch. In preferred embodiments, the additive is one or a mixture of two or more of kaoline, silk amino acid, silicone dioxide, chitosan. In further preferred embodiments, the additive comprises a food grade or natural bioderived additive such as any cellulose or starch derived from seaweed or other sources, other seaweed extracts such as proteins, polyphenols or fucoidans, high or low methoxyl pectin (LMP/HMP), dextrose, fructose, natural clays with a particle size less than 30 microns (preferably between 5-10 microns), polyphenols such as tannic or gallic acid, proteins such as zein, chitosan. The additives may also comprise waste materials and extracts derived from the seaweed extraction process, such as waste seaweed fibers, filtration agents or waste fruit and vegetable peels, including those with high polyphenol content such as grapes, honey, mango, blueberries, pomegranates, or apple.

In some embodiments, the membrane comprises an additive to improve compression strength of the membrane, such as konjac glucommanan or a sugar alcohol (such as mannitol, xylitol, sorbitol). Preferably, the sugar alcohol is mannitol, as it offers the most improvement to the membrane.

In some embodiments, the membrane comprises an additive to improve permeability and brightness, such as low methoxy pectin, preferably variations which contain a low quantity of calcium salts.

The membrane mixture may be blended with a plasticising additive in order to increase plasticity prior to injection. Therefore, in some embodiments, the additive comprises a plasticising additive. Suitably, the plasticising additives may comprise glycerol, sodium stearate, agar, carrageenan or pectin, or a mixture or two thereof. In addition, pectin can be added to make the membrane wall of the pearl visually clearer and brighter. The same effect can be observed with the addition of other simple sugars such as fructose or dextrose which can also be added to improve permeability of the membrane. Therefore, in some embodiments, the membrane further comprises pectin or a simple sugar such as fructose or dextrose, or a mixture of two or more thereof.

Examples of other plasticisers include Dihydric acids (Ethylene glycol, propylene glycol), Trihydric alcohols (Glycerol, polyglycerol), Sugar alcohols (Sorbitol, mannitol, maltitol, xylitol), Saccharification products of reduced, starch (glucose, fructose, galactose andxylose), Disaccharides (saccharose, maltose and lactose), Amine acids (in combination with polyols e.g. Proline, glycine, anilin), Fatty acids-short chains (Capric acid, caproic acid).

Thickening agents as described above can also be added to the alginate membrane to improve processability, mechanical strength and permeability. In preferred embodiments, such thickening agents comprise one or a mixture of two or more of guar gum, low/high methoxyl pectin, konjac glucommanan, preferably konjac glucommanan which is available in different viscosities, and can thus be tuned to enable better processability within the injection machine.

The membrane may also include a natural or bioderived surfactant to enhance or reduce elasticity of the membrane as required. Surfactants can also be used as a wetting agent to increase or decrease the interfacial surface tension of the membrane mixture and different content materials, or as an emulsifier to enable the incorporation of hydrophobic ingredients into the membrane or contents.

Hydrophile Lipophile Balance (HLB) is a way of measuring a substances solubility within water or oil. The HLB characterisation system is used to identify the correct type of surfactant for a given application. The HLB system enables the user to assign a HLB value to a surfactant and a HLB requirement to the application for that surfactant. Properly matching these values helps develop appropriate formulations for each application. For example, high HLB surfactants can be used to increase hydrophilicity of a membrane mixture and to stabilise oils (when incorporated into the membrane or the contents). Mid HLB surfactants above 10 e.g. sorbitan esters are more hydrophilic.

Anionic surfactants may improve elasticity in the membrane. Therefore, in some embodiments, the membrane comprises one or a mixture of two or more anionic surfactants such as sodium stearate or sodium lauryl sulfate.

Nonionic surfactants may reduce elasticity. Therefore, in some embodiments, the membrane comprises one or a mixture of two or more nonionic surfactants, such as lauryl glucoside, decyl glucoside, glycol distearate, glycerol monostearate, propylene glycol isostearate, a Tween® (such as Tween®20, 40, 60, 80), a Span™ (such as Span™ 20, 40, 60, 80, 85) or a Polysorb® (such as Polysorb®85/70/00), polysorbate (such as polysorbate 20, 40, 60, 80) or lecithin.

In some embodiments, the thickener, additive, or diluent that may be present are selected to have a neutral odour and/or taste. Using a mixture with neutral taste and/or odour has the advantage of avoiding taste contamination of the product once encapsulated.

The addition of a food additive, food flavouring or fragrance to the viscous liquid may be for the purposes of creating a more desirable consumer product, for example to enhance the taste or smell of it. Suitable food additives, food flavourings and fragrances include natural occurring sugars such as dextrose or fructose.

A preservative is a substance or a chemical that is added to products such as food products, beverages, cosmetics, and many other products to prevent decomposition by microbial growth or by undesirable chemical changes. Suitable preservatives include a mixture of citric acid and potassium sorbate, a mixture of lactic acid and potassium sorbate, or a mixture of ascorbic acid and potassium sorbate. In preferred embodiments, the preservative comprises citric acid and potassium sorbate, as this mixture has the least effect on flavour and cost.

In further preferred embodiments, the preservative comprises a naturally derived preservative such as fucoidans derived from seaweed, mushroom extracts, rosemary and oregano extract, or diatomaceous earths.

In some embodiments, the preservative does not comprise sodium benzoate.

Diluents may be used to reduce the viscosity of the viscous liquid. Suitable diluents include plasticisers such as glycerol or propylene glycol.

In some embodiments the concentration of calcium in the membrane mixture (i.e. In the mixture comprising alginate which is used to form the membrane) is 0.02% or less, such as less than 0.015%, 0.01%, 0.005% or 0.001%. In some embodiments the mixture comprising alginate which is used to form the membrane is substantially free of calcium. In some embodiments the mixture is free of calcium.

In some embodiments the concentration of protein in the membrane mixture (i.e. in the mixture comprising alginate which is used to form the membrane) is 50% or less, preferably 30% or less, more preferably 20% or less, even more preferably 10% or less, such as 5% or less, for example 2% or less, such as less than 1.5%, 1% or 0.5%. In some embodiments the mixture comprising alginate which is used to form the membrane is substantially free of protein. In some embodiments the mixture is free of protein.

In some embodiments, the viscous liquid and/or the membrane are edible or suitable for applying onto a body part.

Suitably, the viscous liquid may be edible and comprise, such as consist of, various food and drink related products. In accordance with the disclosure herein, the skilled person will understand that only products that have a water content of between 2 wt % and 55 wt % (such as 5 wt % and 55 wt %) are suitable. Suitably the viscous liquid comprises greater than 80 wt % of a food or drink product, preferably greater than 85 wt %, preferably greater than 90 wt %, more preferably greater than 95 wt %, such as greater than 98 wt %, most preferably 100 wt %. Suitable food and drink related products are herein described below (but not limited to):

Sauces and condiments, which are shear-thinning non-Newtonian fluid food compounds typically comprising salt, sugar and one or more spices which when added to a food enhances the flavour of the food. In some embodiments, the food or drink product is a sauce or condiment.

Energy gels are a class of low water content carbohydrate rich gels, typically comprising a blend of sugars, most often maltodextrin and fructose, caffeine and preservatives. In some embodiments, the food or drink product is an energy gel.

In some embodiments, the food or drink product is coffee, baby formula, baby feeds, yogurt, cream cheese, smoothies, protein shakes, or protein gels, preferably coffee.

Suitably, the viscous liquid may be non-edible and may comprise, such as consist of, personal hygiene and cosmetic products. In accordance with the disclosure herein, the skilled person will understand that only products that have a water content of between 2 wt % and 55 wt % (such as 5 wt % and 55 wt %) are suitable. Suitably the viscous liquid comprises greater than 80 wt % of a food or drink product, preferably greater than 85 wt %, preferably greater than 90 wt %, more preferably greater than 95 wt %, such as greater than 98 wt %, most preferably 100 wt %.

Personal hygiene and cosmetic products are constituted mixtures of chemical compounds derived from either natural sources or synthetically created ones, designed for personal care and skin care, and can be used to cleanse or protect the body or skin. Suitably the viscous liquid comprises greater than 80 wt % of a personal hygiene and cosmetic product, preferably greater than 85 wt %, preferably greater than 90 wt %, more preferably greater than 95 wt %, such as greater than 98 wt %.

Personal hygiene and cosmetic products suitable for use in the present invention include cleansers, toners, serums, moisturisers, balms, shampoo, conditioner, shower gel, skin creams, face masks, exfoliants, moisturisers, sun creams, hair conditioner, hair gel, hair cream, soap, liquid soap, hand soap, body wash, lip-gloss, foundation, liquid lipstick, liquid eyeshadow. Preferably the personal hygiene and cosmetic product is one of shower gel, shampoo, conditioner, sun cream, skin care cream or toothpaste.

Shower gel, shampoo, conditioner and liquid soap are viscous liquids used for cosmetic and hygiene applications typically comprising surfactants, foaming agents, conditioners, thickeners, opacifiers, sequestering agents, preservatives, special additives, and/or fragrance. In some embodiments, the personal hygiene and cosmetic product is a liquid soap, face mask, shower gel, shampoo or conditioner.

Sun creams comprise an active ingredient intended to protect the skin from the sun. In some embodiments, the personal hygiene and cosmetic product is sun cream.

Toothpaste is a paste or gel dentifrice typically comprising glycerin to stop the paste from drying out, calcium carbonate which acts as an abrasive, sodium lauryl sulfate which acts as a detergent and makes toothpaste foam, sodium saccharin for flavour, and fluoride to strengthen tooth enamel. In some embodiments, the personal hygiene and cosmetic product is toothpaste.

In some embodiments, the viscous liquid does not comprise any anionic surfactant, and/or cationic surfactant, and/or sodium chloride, and/or cross-polymer.

Anionic surfactants are surfactants that carry a negatively charged head group. Anionic surfactants include ammonium lauryl sulfate, sodium laureth sulfate, sodium lauryl sarcosinate, sodium myreth sulfate, sodium pareth sulfate, sodium stearate, sodium lauryl sulfate, a olefin sulfonate, and ammonium laureth sulfate, CITREM (Citric acid esters of mono- and diglycerides of fatty acids, 1,5-dioxo-1,5-bis(3,5,5-trimethylhexylocy)-3-((3,5,5 trimethylhexyloxy)carbonyl)pentane-2-sulfonate).

Cationic surfactants are surfactants that carry a positively charged head group. Cationic surfactants include benzalkonium, benzethonium, methylbenzethonium, cetylpyridinium, alkyl-dimethyl dichlorobenzene ammonium, dequalinium and phenamylinium chlorides, cetrimonium and cethexonium bromides.

The cross-polymer refers to an acrylate, such as a C₁₀₋₃₀alkyl acrylate cross polymer. Suitably the cross-polymer may also include any naturally derived polymer which is not chemically modified as described above.

In some embodiments, the diameter of the water-based viscous liquid encapsulated within a membrane is from 1 cm to 5 cm, preferably 1 cm to 3 cm, such as 1 cm to 2 cm.

In some embodiments, the volume of the viscous liquid encapsulated in the membrane is from 1 to 40 mL, preferably 1 mL to 30 mL, more preferably 2 to 30 mL, more preferably 3 to 25 mL, yet more preferably 5 to 20 mL.

In some embodiments, the volume of the viscous liquid encapsulated in the membrane is at least 1 mL, such as at least 2 mL, 3 mL, 4 mL or 5 mL. In some embodiments, the volume of the viscous liquid encapsulated in the membrane is less than 40 mL, such as less than 35 mL, 30 mL, 25 mL, 20 mL, 15 mL, 12 mL, 10 mL or 8 mL. Any of the aforementioned lower or upper limits of the ranges may be combined with each other, and are herein disclosed.

In some embodiments, the viscous liquid is a single dose. A single dose means a single portion usually for use by a single consumer, for example an amount of toothpaste suitable for a single toothbrushing, or an amount of moisturiser suitable for a single application to the user's face.

The single dose has the advantage of reduced waste and reduced plastic pollution as it would enable consumers to use the appropriate amount of a product (e.g. toothpaste portion or a condiment portion). Additionally, it would enable consumers to only carry with them the appropriate amount of product when traveling, e.g. two pearls of toothpaste for a day of travelling, instead of carrying a whole plastic toothpaste tube, which is generally also not recyclable.

It is an advantage of the invention that the viscous liquid remains stably stored within the membrane. This allows the product contained with the membrane to be stored over a significant period of time. Suitably the viscous liquid remains stably stored within the membrane for at least 1 week, preferably at least 2 weeks, more preferably at least 3 weeks, yet more preferably at least 4 weeks, such as at least 6 weeks or 8 weeks.

The membrane contains the viscous liquid without losing significant amounts of it via permeation. Suitably, the viscous liquid has less than 15 wt % mass loss 24 hours after manufacturing, preferably less than 10 wt % mass loss 24 hours after manufacturing, preferably less than 7 wt % mass loss 24 hours after manufacturing, more preferably less than 4 wt % mass loss 24 hours after manufacturing, as measured by the method described in Example 2

In some embodiments, the membrane is spherical, ovoid or droplet shape. Preferably, the membrane is spherical.

In some embodiments, several pearls are grouped together such as within a second membrane. The second membrane may be a further alginate layer or other suitable hydrogel or protective layer which is peelable or otherwise removable from the product but provides further hygienic protection to the encapsulated product. Such a layer would be similar to that of a fruit skin and designed to allow the consumer to remove it from the product as required. This layer may comprise a wax or fatty acid composition, or a mixture of hydrophobic materials that may act as a further barrier to water permeation. Examples of waxes include beeswax, candelilla wax, carnauba wax, jojoba oil, ouricury wax. Examples of fatty acids include capric acid, laurc acid, myristic acid, palmitic acid or stearic acid. Natural sources of these fatty acids include coconut oil, palm kernel oil, oil palms, coca butter, shea butter.

The exterior of the encapsulated product, comprising alginate cross-linked membrane, whether it comprises a further secondary layer or not, may be a useful substrate to which edible ink or imagery may be applied in the form of text, pictures or logos. Text of a logo can be printed or applied for marketing purposes or hidden and introduced between two layers in the case of a secondary membrane.

In a second aspect, the invention provides a method of preparing a water-based viscous liquid encapsulated within a membrane according to the first aspect, wherein the method comprises the steps of:
(i). providing an aqueous alginate solution free from calcium ions;
(ii). providing a viscous liquid having a water content from 2 wt % to 55 wt % and calcium ions;
(ii). Immersing the viscous liquid of step (ii) in the alginate solution of step (i) thereby forming the water-based viscous liquid encapsulated within a membrane.

The viscous liquid and the membrane may have any of the features described above for the first aspect.

In some embodiments, the concentration of alginate in the alginate solution of step (i) is present in an amount from 0.1 wt % to 10 wt %, preferably from 0.3 wt % to 10 wt %, preferably from 0.5 wt % to 10 wt %, preferably from 0.5 wt % to 8 wt %, more preferably 0.5 wt % to 5 wt %, yet more preferably 0.5 wt % to 3 wt %, such as 1 wt % to 2 wt %.

Suitably, step (iii) is undertaken using any standard mixing equipment e.g. overhead mixer, blender, high sheer mixer, or a vacuum blender.

In some embodiments, calcium salts are added to the viscous liquid of step (ii) In order to provide the calcium ions in the viscous liquid (via dissociation of the calcium salts). Suitably the calcium salts added to the viscous liquid of step (ii) is an amount from 1 wt % to 15 wt %, preferably 1 wt % to 10 wt %, preferably 2 wt % to 10 wt %, more preferably 2 wt % to 8 wt %, more preferably 2 wt % to 5 wt %.

In some embodiments, the immersing in step (Iii) is done by injection dropping the viscous liquid into the alginate solution. Suitably the viscous liquid may be injected into the alginate solution using a syringe (manual or machine operated) or a pump (such as a peristaltic pump), a compressed air powered piston, a vibrating nozzle or other injecting systems using gravity. Suitably the viscous liquid is in droplet or spherical form when it is immersed in the alginate solution.

Suitably immersion of the viscous liquid in the alginate solution is for at least 2 minutes. This allows the outer layer of the viscous liquid to react forming the calcium Ion cross linked alginate membrane. Suitably the immersion time can also be tuned to be less or more depending on the desired thickness or strength of the membrane wall. A longer time will result in a thicker membrane, less time results in a thinner membrane. This may be desirable for certain applications, for example, a thinner membrane will be less strong and so may be better for edible applications. Thicker membranes will be better for longer term storage applications for added durability and reduced mass loss.

Suitably the membrane will have a thickness of 0.2 mm to 2 mm, preferably 0.3 mm to 1 mm, most preferably 0.5 mm to 0.7 mm.

Suitably the viscous liquid is provided as a predetermined dosage.

In some embodiments, after formation of the viscous liquid encapsulated within a membrane and its removal from the alginate bath, it is thoroughly drained to remove the excess alginate.

In some embodiments, the method further comprises a step (iv) of immersing the formed water-based viscous liquid encapsulated within a membrane in a solution of calcium salt or water, preferably calcium salt. This results in additional cross-linking to form on the external side of the alginate membrane making it stronger and less permeable. Suitably the immersion occurs for at least 20 seconds, preferably at least seconds. Longer immersion time in the calcium bath creates a stronger membrane. Shorter time creates a weaker membrane which may be desirable for easy popping/easy dissolving in the mouth or on application to the body. Suitably the concentration of calcium salt in the solution is between 1 wt % and 15 wt % preferably 1 wt % to 10 wt %, preferably 2 wt % to 10 wt %, more preferably 2 wt % to 8 wt %, more preferably 2 wt % to 5 wt %. In one aspect the calcium salt concentration is a low concentration (such as 1 wt % to 3 wt %), as it is desirable for edible applications that the calcium concentration is low otherwise the taste will be affected.

In some embodiments, the method may comprise a further step of applying a second membrane around the membrane of the encapsulated viscous liquid. The second membrane may also comprise alginate and calcium ions. The second membrane reduces the water permeability.

Additives can be added to the membrane to provide additional functional/performance effects e.g. increased mechanical strength or improved permeability to water. Thus suitably the alginate solution further comprises an additive as described earlier in the specification, preferably the additive is silicone dioxide, kaoline, annatto, bamboo fibre, silk amino acid, or chitosan.

In some embodiments, the method may comprise a further step (v) of treating the membrane with UV radiation (30 seconds, 254 nm) or boiling (for minimum 10 seconds by complete immersion or blanching). The inventors have found this to reduce water permeability and enhance mechanical strength.

In specific embodiments, the viscous liquid of step (ii) is pumped through a peristaltic pump or piston into a bath of alginate solution described in step (i) to deliver a set content dosage. In some embodiments, the viscous liquid comprises a cosmetic product and the dosage is from 5 ml to 30 mL. In alternative embodiments, the viscous liquid comprises toothpaste and the dosage is from 1 mL to 5 mL. In alternative embodiments, the viscous liquid comprises a sauce or condiment and the dosage is from 2 mL to 20 mL. In alternative embodiments, the viscous liquid comprises energy gel or coffee and the dosage is from 5 mL to 20 mL.

In some embodiments, the method comprises the further steps of washing, and/or draining and/or packing of the pearls formed in steps (iii) or (iv). Suitably the packing may be packing single or multiple pearls into a sealed container, such as a glass jar.

In some embodiments, the method comprises the further step of taking two or more, such as three, of the previously formed alginate pearls and dipping them in an alginate solution whilst maintaining contact between the pearls to agglomerate them in a group.

In some embodiments, the method comprises the further step of taking a pearl formed in steps (iii) or (iv) and submerging it in heated water at a temperature above 80 degree C. Suitably the pearl remains in the water for 1-30 minutes.

Optionally the water is gently stirred. This allows the pearl to continue to rotate and heat uniformly. Suitably the pearl is then removed from the water.

Subsequent post-processing methods may be used to reduce the deflation or appearance of water loss from the pearl, such as tumble drying (moderate heating and tumbling inside a metal frum, to reduce excess moisture). In some embodiments, the method comprises a subsequent step of reducing excess moisture, such as tumble drying.

In a third aspect, the invention provides the use of the water-based viscous liquid encapsulated within a membrane as provided in the first aspect or the container of the fourth aspect to:
(i). package and/or store the viscous liquid; and/or
(ii). transport the viscous liquid; and/or
(iii). provide a single dose of the viscous liquid to a user.

The third aspect of the invention may have any of the features described above for the first and second aspects of the invention.

In a fourth aspect, the invention provides a container comprising (such as consisting of) at least one water-based viscous liquid encapsulated within a membrane according to the first aspect of the invention. The container acts as secondary packaging for storing and transporting the pearls of the invention.

The container may be made of a reusable or recyclable material. The container may be a glass jar, or a cardboard box or tube.

The water-based viscous liquid encapsulated within a membrane of the first aspect of the invention may be packaged in a container for storage or transport, without the need to submerge the pearls in liquid. Accordingly, suitably the container does not comprise further liquid.

The invention will now be described by way of the following non-limiting examples.

The skilled person will understand that features which are optional can be used in different combinations to construct various different embodiments and examples of the invention not limited to those shown herein.

### EXAMPLES

### Example 1—Preparation of Pearls Containing Coffee

### Method of Preparation

An aqueous non-calcium alginate solution in which the concentration of alginate within the range 0.5% to 2% by weight was prepared.

A low water content coffee solution to be encapsulated was then prepared by adding 5% calcium salt (as a source of calcium ions) by weight of the coffee solution. This coffee solution was then pumped through a peristaltic pump into a bath of the alginate solution to deliver set content dosage within the range of 5 ml to 20 ml. The coffee pearls thus obtained were immersed for 2 minutes to allow the outer layer of the pearl to react forming a calcium ion cross linked alginate membrane.

The pearls were thoroughly drained of excess alginate and then immersed into a bath of calcium salt solution in which the concentration of calcium salt was 5% by weight of the solution for 30 seconds to complete external cross linking of alginate membrane. The pearls were then washed, drained and packaged. Pearls of various diameters (2-10 mm) were made.

### Permeability Assessment

The coffee syrup was mixed with water and glycerol giving solutions of different water concentrations. As shown in Table 1, the coffee pearls which had less than 55 wt % of water content showed little to negligible % mass loss after 24 hrs (permeability). The control composition, which had 97 wt % water, had poor permeability (noticeable mass loss >15%) after 24 hours.

The concentration of the alginate bath used was changed for each content dependent on the size of the pearl produced (1 ml, 4 ml & 8 ml). This is because the glycerol increases the density of the coffee content and therefore thicker alginate solutions (high concentration of alginate) are required for contents containing more glycerol to allow sufficient time for pearl droplet formation in bath. If the concentration is too high the pearl will not sink, if the concentration is too low the pearl will sink too fast and form a puddle at the bottom of the alginate bath.

The following method was used to determine permeability, as a wt % mass loss over time. The pearl was placed in a storage jar with an airtight lid, and the jar with pearl inside was then weighed, recording this as the initial mass. The jar was then stored in the fridge, or room temperature if performing ambient temperature permeability. The weight was then measured after a period of time, e.g. 24 hours, 48 hours, 72 hours and 96 hours by removing the jar from its storage location, opening the lid, drying any excess moisture from the pearl surface or from inside the jar and then subsequently weighing the pearl inside the jar. The resulting mass difference can then be reported as a % mass loss over time, and acts as an indication of the pearl permeability performance.

TABLE 1
Permeability assessment

| | Encapsulated content | Mass loss over 24 |
|---|---|---|
| composition | composition | hrs (Permeability) |
| A | 3% coffee wt, 84.9% | Negligible <1% |
| | glycerol, 7.4% water | |
| B | 5.5% coffee wt, 76.4% | Negligible <1% |
| | glycerol, 13.6% water | |
| C | 7% coffee wt, 71.3% | Negligible <1% |
| | glycerol, 17.3% water | |
| D (50/50 glycerol/water) | 7% coffee wt, 44.3% | Minimal <5% |
| | glycerol, 44.3% water | |
| Control (Alginate bath | 3% coffee wt, 97% water | Noticeable mass |
| 1% conc; 5% calcium | | loss >15% |
| gluconolactate) | | |

### Example 2—Further Treatment of the Membrane

### Method

Pearls in Table 2 below were formed according to the method described in Example 1, the encapsulated content was as described for Composition A in Table 1 above. For those examples in Table 2 which contain an additive in the membrane, e.g. Process B, C, D, E, G, H, I, the additive was added to the alginate membrane mixture in the bath in the desired concentration to improve the pearl performance. For the processes described as treatments (process A, process F) these were performed after formation of the pearl and after cross linking. The pearls were thoroughly drained of excess alginate and then immersed into a bath of calcium salt solution in which the concentration of calcium salt was 5% by weight of the solution for 30 seconds to complete external cross linking of alginate membrane. The pearls may be left in this solution, or a weaker calcium solution e.g. tap water for a longer length of time to improve the mechanical strength of the membrane. The pearls were then washed, drained and processed via the treatment details given in Table 2. The control pearl had a standard alginate/calcium membrane with no additive or additional treatment and it also had the encapsulated content as described for Composition A in Table 1 above.

TABLE 2
additional process treatments and results

| | Mass loss (permeability) |
|---|---|
| | % better/worse compared |
| Process (Additive / Treatment) | to control after 24 hrs |
| Process A (Treatment: UV 254 nm, 30 | 22% better |
| seconds treatment) | |
| Process B (Additive: 0.22% Silicone dioxide) | 12.5% better |
| Process C (Additive: 0.2% Kaoline) | 14% better |
| Process D (Additive: 0.2% Annatto) | 9% better |
| Process E (Additive layer: Double coating) | 12% better |
| Process F (Treatment: Immersion in boiling | 21% better |
| water Boiling for minimum 5 seconds | |
| treatment) | |
| Process G (Additive: 0.15% Bamboo fibre) | 8% better |
| Process H (Additive: 0.2% Silk amino acid) | 12% better |
| Process I (Additive: 0.4% Chitosan) | 16% better |

### Example 3—Pearls Containing Energy Gel

A pearl (diameter 2.5 cm) containing energy gel was prepared following the method in Example 1. The water content of the energy gel was 25-30%. It was then stored in a closed jar at ambient temperature with a minimum shelf life of 1 month.

Water permeation over time was measured and the results are shown in Table 3. The water permeation observed was partly water of the alginate membrane drying out, which explains the initial decline and then plateau for the pearls showing there is no product leakage over time.

TABLE 3
water permeation over time

| Time | Mass loss over time |
|---|---|
| 24 hours | Minimal |
| 48 hours | None |
| 72 hours | None |

Compression resistance was also measured over time. and the results are shown in Table 4. The compression resistance of the energy gel pearls improves over time as the membrane becomes fully crosslinked and dries. This assists with the ability of the pearl to remain stable over long term storage.

TABLE 4
compression strength over time

| Time period after which | |
|---|---|
| compression strength | |
| was measured | Compression strength |
| 0 hours | Acceptable |
| 24 hours | Good |
| 48 hours | Very good |

### Example 4—Pearls Containing Personal Hygiene and Cosmetic Products

Toothpaste, sun cream, lipgloss, shampoo, conditioner, hand wash, body wash pearls with water contents of less than 50 wt % were also successfully made using the process described in Example 1.

Colgate toothpaste pearls, wherein the toothpaste has 5-10 wt % water content, and sun cream wherein the water content was 30-45 wt % were prepared and observed to have a shelf life of at least 6 months; no degradation or permeated toothpaste/sun cream was observed

### CONCLUSION

Viscous liquids with a water content from 2 wt % to 55 wt % have been successfully prepared in various sizes, including sizes appropriate for single dose use. Both edible and non-edible products within the pearls have been made and have been found to be stable over time. Thus they are useful for long-term packaging and storage of consumer products. Advantageously, the pearls as disclosed herein offer an environmentally friendly alternative to the currently widely used single-use and non-biodegradable or non-recyclable plastic packaging. This can help the ever increasing issues relating to climate change and environmental damage caused in part by plastic packaging.

### Example 5: Groupings

For certain applications it preferable that several pearls are grouped together within a secondary layer in order to create a peelable hygienic layer similar to that of a fruit skin, revealing individual sip size containers or compartments, like segments in an orange that can then be separated for use.

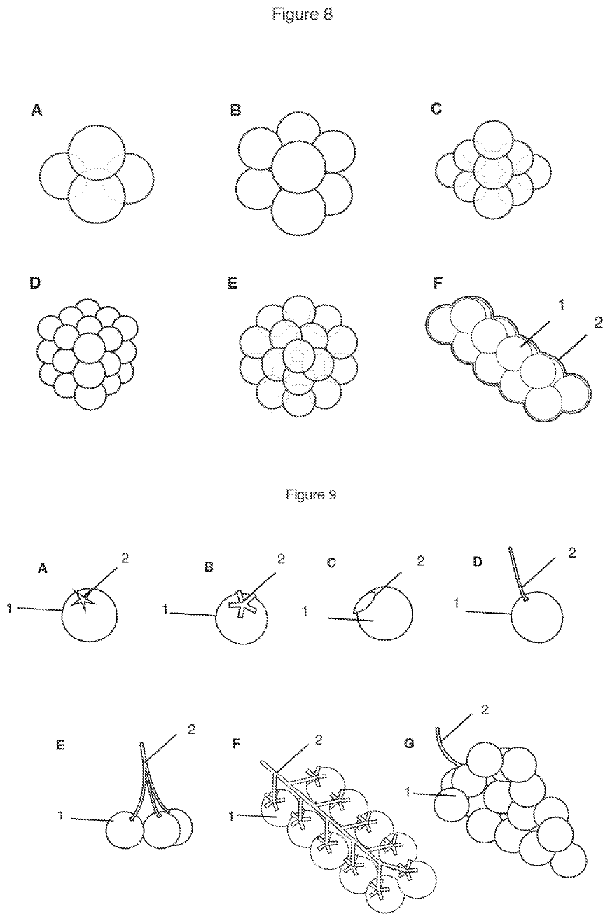

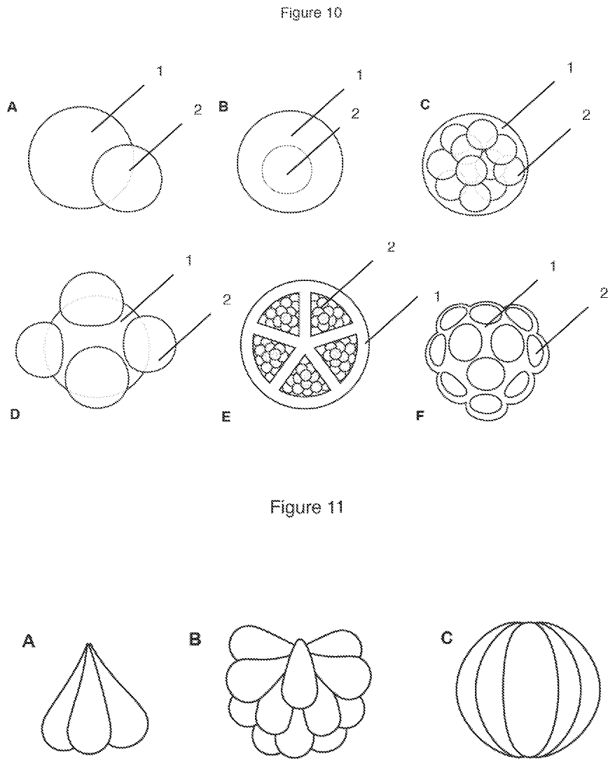

Such groupings can be created with the following steps: A thick solution of 1-10% of sodium alginate was prepared at room temperature, typically 2%, with a viscosity in the range of 50-5000 cps. Optionally colorant was added. Once the solution was homogeneous and no air bubbles remain, the previously formed alginate pearls were dipped in the solution, maintaining contact between them to agglomerate them in a group. They were then delicately extracted from the solution and excess solution let to drip, forming a homogeneously thick layer of solution at the surface of the containers. The concentration of alginate and the time left for dripping define the thickness of the secondary layer. The grouping was then submerged in a bath of 1 to 10% Calcium ions, typically calcium chloride at 2% and let to crosslink for 1-30 min. The grouping was then removed from the bath and dried before being ready to be used. The process can be repeated as many times as required to increase the mechanical resistance and reduce permeability of grouping. Examples are shown in FIGS. 7-11.

### Optional Processing Techniques

Temperature Hardening: It is preferred for the membrane of the alginate groupings to be dense and of low permeability in order to improve the packaging functionality. It is also important that their appearance is smooth and aesthetically pleasing. Improving these properties is possible by forming the alginate pearls, as provided in one of the previous examples, and submerging in heated water at a temperature above 80 degree C. The alginate containers remained in the water for 1-30 minutes. Optionally the water is gently stirred so that the alginate containers continued to rotate and were heated uniformly. The containers were extracted from the water, cooled and dried. The membrane of the container post treatment was denser, harder and shinier.

### Example 7—Further Comparative Example

| | Total water content | Mass loss (%) |
|---|---|---|
| Encapsulated liquid | (wt %) | over 24 hours |
| Sugar syrup, natural | 65% | 30% |
| flavourings (84%), added | | |
| water 20% | | |

Pearls containing a high water content (e.g. 65%) are less stable than pearls containing lower water content, as demonstrated by the total water loss of >30%.

### Example 8—Membrane Compositions

Pearls with a membrane composition comprising an emulsion with alginate (2-4%) and oil (7-12%) and natural surfactant (0.01-0.1%) were found to have 50% reduced water loss compared to standard membrane. Lower viscosity membrane formulations work better with higher viscosity contents.

1. A water-based viscous liquid encapsulated within a membrane, wherein the viscous liquid has a water content from 2 wt % to 55 wt %, and wherein the membrane comprises alginate and calcium ions.

2. The water-based viscous liquid encapsulated within a membrane according to claim 1, wherein the viscous liquid has a viscosity from 1 to 100,000 cps, more preferably 1 to 20,000 cps, most preferably 100 to 4,000 cps.

3. The water-based viscous liquid encapsulated within a membrane as claimed in either claim 1, wherein the viscous liquid has a water content from 5 wt % to 55 wt %, preferably from 5 wt % to 50 wt %, more preferably from 5 wt % to 45 wt %, yet more preferably from 5 wt % to 40 wt %, even more preferably from 5 wt % to 35 wt %, still more preferably from 5 wt % to 30 wt %, such as from 5 wt % to 25 wt %, or from 5 wt % to 20 wt %.

4. The water-based viscous liquid encapsulated within a membrane as claimed in claim 1, wherein the viscous liquid comprises a thickening agent, optionally wherein the thickening agent comprises starch, modified starch, cellulose gel, guar gum, tara gum, carrageenan, gum tragacanth, locust bean gum, microcrystalline cellulose, pectin, gellan gum, glucumannans, succinoglucan, konjac glucomannan, agar, pectin, xanthan gum, guar gum, gum arabica, glycerol, or mixtures of two or more thereof, preferably glycerol.

5. (canceled)

6. The water-based viscous liquid encapsulated within a membrane as claimed in anyone of the claim 1, wherein the calcium ions are sourced from one of calcium gluconolactate, calcium chloride, calcium iodide, calcium bromide, calcium fluoride, calcium hydroxide, calcium citrate, calcium acetate, calcium gluconate, or calcium carbonate, or a combination of two or more thereof, preferably calcium gluconolactate and/or calcium chloride.

7. The water-based viscous liquid encapsulated within a membrane as claimed in claim 1, wherein the viscous liquid comprises a non-ionic surfactant and/or an amphoteric surfactant, optionally wherein the non-ionic surfactant comprises one or more of lauryl glucoside, decyl glucoside and/or the amphoteric surfactant comprises cocoamidopropyl betaine.

8.-9. (canceled)

10. The water-based viscous liquid encapsulated within a membrane as claimed in claim 1, wherein the membrane comprises an additive, preferably the additive is silicone dioxide, kaoline, annatto, bamboo fibre, silk amino acid, or chitosan.

11. The water-based viscous liquid encapsulated within a membrane as claimed in claim 1, wherein the membrane is continuous.

12. The water-based viscous liquid encapsulated within a membrane as claimed in claim 1, wherein the viscous liquid and/or membrane is edible or for applying onto a body part.

13. The water-based viscous liquid encapsulated within a membrane as claimed in claim 1, wherein the viscous liquid comprises i) a personal hygiene or cosmetic product, preferably shower gel, shampoo, conditioner, sun cream, skin care cream, or toothpaste, or (ii) a food or drink product, preferably a sauce, condiment, energy gel or coffee, optionally wherein the viscous liquid comprises greater than 80 wt % of the personal hygiene or cosmetic product or the food or drink product, preferably greater than 85 wt %, preferably greater than 90 wt %, more preferably greater than 95 wt %, such as greater than 98 wt %.

14. (canceled)

15. The water-based viscous liquid encapsulated within a membrane as claimed in claim 1, wherein the viscous liquid does not comprise anionic surfactant, and/or cationic surfactant, and/or sodium chloride, and/or cross-polymer.

16. The water-based viscous liquid encapsulated within a membrane as claimed in claim 1, wherein the membrane encapsulates from 1 to 40 mL of the viscous liquid, preferably 2 to 30 mL, more preferably 3 to 25 mL, optionally wherein the viscous liquid is a single dose.

17. (canceled)

18. The water-based viscous liquid encapsulated within a membrane as claimed in claim 1, wherein the viscous liquid remains stably stored within the membrane for at least 1 week, preferably at least 2 weeks, more preferably at least 3 weeks, yet more preferably at least 4 weeks.

19. The water-based viscous liquid encapsulated within a membrane as claimed in claim 1, wherein the viscous liquid has less than 15 wt % mass loss 24 hours after manufacturing, preferably less than 10 wt % mass loss 24 hours after manufacturing, preferably less than 7 wt % mass loss 24 hours after manufacturing, more preferably less than 4 wt % mass loss 24 hours after manufacturing.

20. A method of preparing the water-based viscous liquid encapsulated within a membrane as claimed in claim 1, said method comprising the steps of:
(i) providing an aqueous alginate solution free from calcium ions;
(ii) providing a viscous liquid having a water content from 2 wt % to 55 wt % and calcium ions;
(iii) immersing the viscous liquid of step (ii) in the alginate solution of step (i) thereby forming the water-based viscous liquid encapsulated within a membrane, optionally by injection dropping the viscous liquid into the alginate solution.

21. The method of claim 20, wherein the concentration of alginate in the alginate solution of step (i) is present in an amount from 0.1 wt % to 10 wt %, preferably from 0.3 wt % to 10 wt %, preferably from 0.5 wt % to 10 wt %, preferably from 0.5 wt % to 8 wt %, more preferably 0.5 wt % to 5 wt %, yet more preferably 0.5 wt % to 3 wt %, such as 1 wt % to 2 wt %.

22. The method claim 20, wherein the calcium salts are present in the viscous liquid of step (ii) in an amount from 1 wt % to 10 wt %, preferably 2 wt % to 8 wt %, more preferably 2 wt % to 5 wt %.

23. (canceled)

24. The method of claim 20, further comprising a step (iv) of immersing the formed water-based viscous liquid encapsulated within a membrane in a solution of calcium salt; and/or step (v) of treating the membrane with UV radiation or boiling.

25. The method of claim 20, wherein the alginate solution further comprises an additive, preferably the additive is silicone dioxide, kaoline, annatto, bamboo fibre, silk amino acid, or chitosan.

26. (canceled)

27. A container comprising at least one water-based viscous liquid encapsulated within a membrane as claimed in claim 1, optionally wherein
(i) the container is made from a reusable or recyclable material;
(ii) the container is a glass jar, or a cardboard box or tube; and/or
(iii) the container does not comprise further liquid.

28.-31. (canceled)
